(19) United States
(12) Patent Application Publication
(10) Pub. No.: US 2023/0372147 A1
(43) Pub. Date: Nov. 23, 2023

(54) A PORTABLE EMERGENCY BLANKET

(71) Applicant: TSG ASSOCIATES LLP, Halifax, Yorkshire (GB)

(72) Inventors: Colin SMART, Halifax, Yorkshire (GB); Simon E. PENNELLS, Halifax, Yorkshire (GB)

(21) Appl. No.: 18/247,995

(22) PCT Filed: Oct. 6, 2020
(86) PCT No.: PCT/GB2020/052462

§ 371 Date: Apr. 5, 2023

(51) Int. Cl. A61F 7/02

(52) U.S. Cl. CPC A61F 7/02; A61F 2007/0018

(57) ABSTRACT

A portable emergency blanket for providing thermal insulation to a patient is disclosed including a cover that houses a first insulating portion, the first insulating portion being configured to insulate a patent's legs in use; and a second insulating portion, the second insulating portion being configured to insulate a patient's torso in use, and the second insulating portion is configured to provide greater insulation than the first insulating portion.

### FIELD

The invention relates to a portable emergency blanket for providing thermal insulation to a patient. More specifically, the present invention relates to a portable emergency blanket for preventing and treatment of hypothermia in a patient. Even more specifically, the present invention relates to a portable emergency blanket for preventing and treatment of hypothermia in a patient situated in a combat environment and/or remote environment.

### BACKGROUND

Hypovolaemic shock caused by haemorrhaging may have disastrous consequences for a person. Initially, the body compensates for the loss of circulating blood volume, but as haemorrhaging continues these compensatory mechanisms fail and the person's condition deteriorates with time.

Hypovolaemic shock may initiate a lethal triad of coagulation, acidosis and hypothermia. Or put another way, severe blood loss results in decreased coagulation, which thereby increases the amount of lactic acid in the blood, which decreases heart performance and leads to hypothermia, which in-turn leads to a further decrease in coagulation. This lethal triad if left untreated results in death.

Hypothermia is traditionally treated broadly using two types of heating techniques. One heating technique is passive heating, which uses the patient's own heat to assist in the reheating process, for example, through the use of towels or additional clothes. Secondly, active heating uses external heat to assist in the reheating process. Active heating is particularly important for seriously injured patients who have lost the ability to produce heat.

Commonly available passive heating equipment for treating hypothermia include canvas stretchers with large amounts of insulation material inside. The insulating material used is commonly a heavy blanket or heavy piece of material which can be wrapped around the patient. To provide greater insulation to a patient's torso the blanket is either wrapped around the patient multiple times or doubled up on itself to provide a plurality of layers across the torso of the patient. The user may wrap the blanket too tightly around the patient or may simple provide too much or too little insulation to a particular region of the patient's body. Furthermore, such blankets are commonly bulky and heavy and are therefore often impractical for groups such as the military and rescue teams where speed is of the essence. Therefore, the blankets aren't considered portable.

It is therefore an object of aspects of the present invention to address one or more of the above-mentioned or other problems.

### SUMMARY

According to the first aspect of the invention, there is provided a portable emergency blanket for providing thermal insulation to a patient, the blanket comprising: a cover that houses: a first insulating portion, the first insulating portion being configured to provide thermal insulation to a patent's legs in use; and a second insulating portion, the second insulating portion being configured to provide thermal insulation to a patient's torso in use; wherein the second insulating portion is configured to provide greater insulation than the first insulating portion.

The portable emergency blanket advantageously insulates parts of the patient's body to varying degrees. The second insulating portion is configured to provide greater insulation to the patient's torso. In contrast, the first insulating portion is configured to provide less insulation compared with the second region to the patient's legs. Tailoring the amount of insulation provided by the blanket at specific locations of the patient's body reduces the amount of material used in the blanket and consequently allows for a small and lightweight blanket.

The first and second insulating portions each comprise an insulating material arranged in one or more layers within the cover, and wherein the second insulating portion has a greater number of insulating material layers than the first insulating portion.

The first insulating portion and/or the second insulating portion may comprise continuous filament fibres.

Suitably, the insulating material used in the first and/or second insulating portions is composed of synthetic materials, such as Climashield®. Advantageously synthetic fibres tend to retain their insulative properties when wet compared with natural synthetic fibres.

The first insulating portion may comprise two layers of insulating material and the second insulating portion may comprise five layers of insulating material.

Having a greater number of insulating material layers in the second insulating portion, compared with the first insulating portion, provides greater insulation to the patient's torso relative to the patient's legs. As discussed above, this reduces the amount of material used in the blanket and consequently allows for a small and lightweight blanket. Furthermore, having multiple insulating material layers in the second insulating portion provides protection from compressive forces arising during transit of the patient. In particular, this is beneficial for military personnel who are often injured in this region of the body.

The insulating material of the first insulating portion and/or the second insulating portion may extend along the width of the blanket. Put another way the insulating material of the first insulating portion and/or the second insulating portion may extend to a periphery on either side of the blanket.

The insulating material of the first insulating portion and/or second insulating portion may be in a substantially uncompressed state when not in use. It is common in commercial quilts to sew multiple pockets/cells into an insulation product. In doing so, the stitching compresses the fibres and thereby reduces the insulation provided by the quilt.

With the present invention, the first and/or second insulating portions may be freely located within the cover. Or put another way, the first and second insulating portions may not be attached to the cover, for example via stitching. Alternatively, the first and/or second insulating portions may be attached, via a periphery, to the cover in order to minimise compression to the insulating portions, in particular, compression to a central region of the insulating portions.

The first insulating portion may have a through thickness of between approximately 12 mm to 18 mm and the second insulating portion may have a through-thickness of between approximately 30 mm to 45 mm.

The cover may comprise at least two flaps on opposing sides of the blanket, each of the flaps comprising a third insulating portion and being operable to be placed around the abdomen of the patient's body.

The third insulating portion may comprise an insulating material arranged in one or more layers within the cover. The third insulating portion may have a greater number of insulating material layers than the first insulating portion. The third insulating portion may comprise five layers of insulating material.

The at least two flaps may be located at or near a periphery of the cover.

The insulating material of the third insulating portion may be in a substantially uncompressed state when not in use.

The third insulating portion has a through thickness of between approximately 30 mm to 45 mm.

Suitably, the insulating material used in the third insulating portion is composed of synthetic materials, such as Climashield®. Advantageously synthetic fibres tend to retain their insulative properties when wet.

The flaps are arranged to be wrapped around the sides of the patient when the patient is lying down. This provides insulation to the sides of the patient without the need to wrap the blanket completely around the patient. In one example, the flaps are configured to reach below the kidney area, but end just proud of touching the ground. In this arrangement, the flaps wrap around the main organs contained in the abdominal space below the rib cage and above the pelvis, but are not compressed under the patient, which would reduce their effectiveness.

The cover may further comprise a neck and/or head flap, the neck and/or head flap comprising a fourth insulating portion.

The fourth insulating portion may comprise an insulating material arranged in one or more layers within the cover. The fourth insulating portion may have a greater number of insulating material layers than the first insulating portion.

The neck and/or head flap may extend at or near a periphery from only one side of the cover. This provides the user with greater flexibility to position the neck and/or head insulating portion.

The insulating material of the fourth insulating portion may be in a substantially uncompressed state when not in use.

The fourth insulating portion has a through thickness of between approximately 30 mm to 45 mm.

Suitably, the insulating material used in the fourth insulating portion is composed of synthetic materials, such as Climashield®. Advantageously synthetic fibres tend to retain their insulative properties when wet.

The neck and/or head insulating portion is arranged to be placed around, for example wrapped around, the neck and/or head of the patient to provide insulation to these areas of the patient.

The blanket may further comprise at least one thermal pad located within the cover, the at least one thermal pad being operable to provide warmth to the patient's body. Suitably, the thermal pad is a thermal gel pad. The thermal pads are operable to provide an active heating source and thereby provide additional heat to the patient. The thermal pads may be located adjacent the first and/or second insulating portions. Alternatively, or in addition to, the thermal pads may be located adjacent the third and/or fourth insulating portions.

The first insulating portion may have a smaller cross-section than the second insulating portion in a direction perpendicular to a longitudinal axis of the blanket. The first insulating portion may be tapered in a direction away from the second insulating portion. This reduces the amount of material used to make the blanket, in particular the first insulating portion, and thereby aids in the production of a small and lightweight blanket.

In one example, the blanket has a width of between approximately 200 mm to approximately 600 mm, more preferably 350 mm. In one example, the blanket has a length of between approximately 1750 mm to 2300 mm, more preferably 2105 mm. In one example, a portion of the blanket is configured to act as a foot flap. For example, a length of up to 400 mm of the blanket is configured to fold under a patient, in use.

In one example, the blanket has a weight of up to up to 100 g, more preferably 50 g.

The blanket may be composed of a polymer material, such as nylon material. In one example, the cover of the blanket in which the insulation is located is formed of a top layer and a bottom layer, wherein the bottom layer is configured to be arranged on the patient, in use. The top layer of the cover may be formed of a water proof material, such as coated nylon material such as 2 oz PU coated nylon ripstop. The bottom layer of the cover may be formed of a material, such as Skylon ultra light showerproof nylon.

According to a second aspect of the invention, there is provided a portable emergency inflatable mattress, the inflatable mattress comprising: a first region to support the patient's head in use; a second region to support the patient's torso and legs in use; the first and second regions comprising a plurality of cells operable to be inflated upon receipt of air to provide a resting surface; and an air valve; wherein the cells in the first region are larger than the cells in the second region.

The mattress is suitably used in combination with the blanket according to the first aspect of the invention, however it may be used with other types of blanket. The mattress and blanket according to the first aspect of the invention may be complimentary in size and/or shape.

The average cross-sectional dimension of the cells in the first region when fully inflated may be between 10 cm to 16 cm, suitably between 12 cm to 13 cm.

The average cross-sectional dimension of the cells in the second region when fully inflated may be between 5 cm to 9 cm, suitably between 7 cm to 8 cm.

The mattress provides insulation to the underside of a patient when lying down on the mattress. The larger cells in the first region of the mattress provide increased comfort and support to the patient's head. Convection inside an air mattress plays an important role in heat transfer away from an individual laid on the mattress. Air is a poor conductor of heat, which is why it is so good to use as an insulator. Convection can play an important role as air turnover within an air-filled mattress can constantly remove heat from the patient's body. Air warmed by the patient's body expands and decreases in density and rises, cooler air within the mattress is denser and sinks, this will naturally create a warmer layer of air next to the patient, the more that these layers mingle and move the lower the temperature is in the air next to the patient.

To minimize the convection turnover within the mattress, certain construction considerations have been made. The first is the ‘egg crate’ overall structure, which creates distinct small pockets of air within the cells of the second region that are not able to mingle with the air in the mattress providing a separate layer of insulation. The relatively low depth and smaller cell size of the cells within the mattress reduce the volume of air minimizing the circulation of the air within the cells.

Suitably, the valve is a one-way valve, such as a Boston Valve.

The valve may be located at the base of the second region, i.e. adjacent to a patient's feet when in use.

The second region of the mattress may be tapered in a direction away from the first region of the mattress. This reduces the amount of material used to make the mattress, and thereby aids in the production of a small and lightweight mattress.

The mattress may be composed of a polymer material, such as a rubber or nylon material. Suitably, the mattress is made from a polyurethane coated nylon material, such as 200HT polyurethane coated nylon material. The 200HT polyurethane coated nylon material may have a yarn of 180 to 220 Denier (0.020 g/m to 0.024), suitably 210 Denier (0.023 g/m).

The mattress may have an outer most width of between 50 cm to 70 cm, suitably between 55 cm to 65 cm, even more suitably 60 cm. The mattress may have a length of between 200 cm to 280 cm, suitably between 230 cm to 250 cm.

In one example, the mattress has a weight of between 0.4 kg to 0.8 kg, more preferably 0.6 kg.

In an embodiment there is provided a method of making a portable emergency inflatable mattress, the method comprising providing a layer of inflatable material, the inflatable material comprising: a first region to support the patient's head in use; a second region to support the patient's torso and legs in use; and an air valve; and heat welding the inflatable material at a plurality of locations to provide a plurality of welding points and a plurality of cells, the plurality of cells being operable to be inflated upon receipt of air to provide a resting surface; wherein the cells in the first region are larger than the cells in the second region.

The heat welding may be spot welding on opposing sides of the inflatable mattress.

The welding points of the first and/or second regions may form a repeating hexagonal pattern. Known mattresses are typically rectangular which means there is excess material that is not touching the body around the top of the head and legs, this material is not providing any insulating benefits to the body. The mattress of the present disclosure is shaped to a typical body shape such that the majority of the mattress is in contact with the body. This keeps the size and weight of the mattress down but does not adversely affect the insulation properties. Other repeating polygonal patterns may be used.

The average distance between adjacent welding points in the first region may be between 5 cm to 9 cm, suitably 8 cm.

The average distance between adjacent welding points in the second region may be between 2 cm to 4 cm, suitably 3 cm.

Suitably, the mattress comprises a heat welded outer periphery.

According to a third aspect of the invention there is provided a survival kit comprising: a holder including a first compartment and a second compartment; a portable emergency inflatable mattress as described previously or a portable emergency inflatable mattress made by the method described previously, the mattress being locatable within the first compartment of the holder; and a portable emergency blanket as described previously, the blanket being locatable within the second compartment of the holder; wherein the holder is operable to be deployed from a carrying configuration to a substantially flat deployed configuration.

The survival kit may further comprise a protection sheet locatable within a third compartment of the holder, the protection sheet being dimensioned to provide an enclosure around the inflatable mattress and blanket when in use by the patient. Suitably, the protection sheet is loose fitting.

The protection sheet may comprise a water resilient material, suitably the water resilient material is a synthetic polymer, such as nylon.

The holder may comprise one or more securing means, the securing means being operable to receive fixtures to secure the holder to the ground when the holder is in the deployed configuration. The securing means may comprise a hole or loop.

The holder may comprise one or more restraining means to restrain the holder in the carrying configuration. The one or more restraining means may comprise a strap. This has the advantage of compressing the holder and contents therein, making the kit easier to carry.

As used herein, unless otherwise expressly specified, all numbers such as those expressing values, ranges, amounts or percentages may be read as if prefaced by the word “about”, even if the term does not expressly appear. The term “about” when used herein means+/−10% of the stated value. Also, the recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Also, any numerical range recited herein is intended to include all sub-ranges subsumed therein.

Singular encompasses plural and vice versa. For example, although reference is made herein to “an” aspirator, “a” breathing da ent” and the like, one or more of each of these and any other components can be used.

The terms “comprising”, “comprises” and “comprised of” as used herein are synonymous with “including”, “includes” or “containing”, “contains”, and are inclusive or open-ended and do not exclude additional, non-recited members, elements or method steps. Additionally, although the present invention has been described in terms of “comprising”, the articles detailed herein may also be described as “consisting essentially of” or “consisting of”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

All of the features contained herein may be combined with any of the above aspects in any combination.

For a better understanding of the invention, and to show how embodiments of the same may be carried into effect, reference will now be made, by way of example, to the following drawings.

### BRIEF DESCRIPTION OF DRAWINGS

FIG. 1 shows a front view of a portable emergency blanket;

FIG. 2 shows a front view of the portable emergency blanket in use with a patient;

FIG. 3 shows a front view of a portable emergency inflatable mattress;

FIG. 4 shows a front view of a protection sheet in use with a patient;

FIG. 5 shows a front view of a holder in a carrying configuration;

FIG. 6 shows a front view of the holder in a deployed configuration;

FIGS. 7a, 7b, 7c and 7d show a perspective view of the portable emergency blanket and portable emergency inflatable mattress is use with patient on a stretcher;

FIG. 8A shows a graph of comfort score (volunteer assessed out of 10) of a volunteer versus time (minutes) for the first test;

FIG. 8B shows a graph of recorded core temperature (degrees Celsius) versus time (minutes) for the first test;

FIG. 9A shows a graph of comfort score (volunteer assessed out of 10) of a volunteer versus time (minutes) for the second test; and

FIG. 9B shows a graph of recorded core temperature (degrees Celsius) versus time (minutes) for the same test.

### DESCRIPTION OF EMBODIMENTS

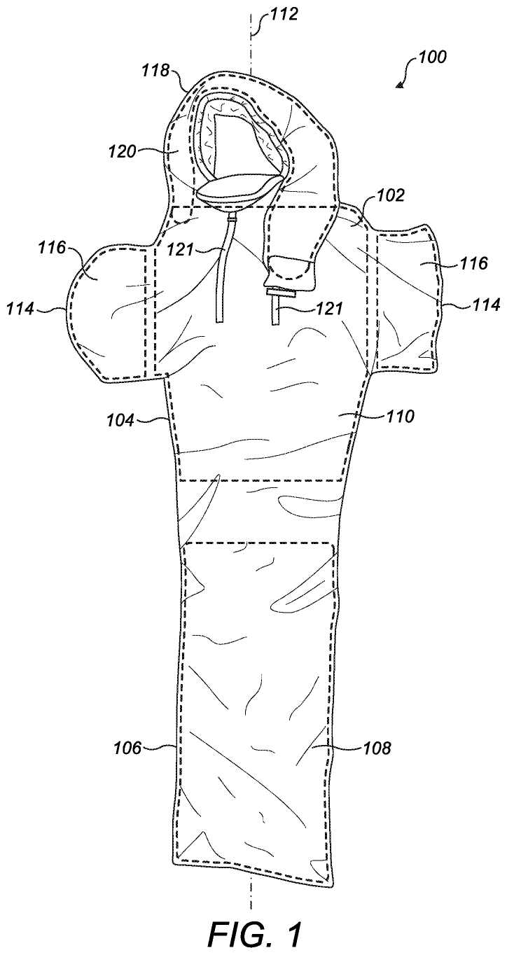

As shown in FIG. 1, a portable emergency blanket 100 for providing thermal insulation to a patient, the blanket 100 comprises a cover 102 that defines a cavity therein.

The cover 102 is the outer surface of the blanket and houses insulation. In one example, the cover 102 is a flexible, lightweight material, such as Crisp 2 oz PU Coated Nylon Ripstop. The cover 102 may provide a sealed cavity.

The cover 102 houses a first insulating portion or area 108 within the cavity. The first insulating portion 108 is configured to provide insulation to a patient's legs, in use. The cover 102 also houses a second insulating portion or area 110 within the cavity. The second insulating portion 110 is configured to provide insulation to a patient's torso, in use. That is to say, that when the blanket 100 is arranged on top of a patient, the first insulating portion 108 is configured to be arranged on a patient's legs and feet and the second insulating portion 110 is configured to be arranged on a patient's torso, i.e. from a patient's shoulders to their groin.

In FIG. 1, the first insulating portion 108 and the second insulating portion 110 are shown schematically. In the example in FIG. 1, there is a short gap between the first insulating portion 108 and the second insulating portion 110, but in other examples, the first insulating portion 108 abuts the second insulation portion 110.

The second insulating portion 110 is configured to provide greater insulation than the first insulating portion 108. That is to say that the blanket 100 works to provide more insulation to the patient's torso compared with the patient's legs.

In one example, both the first insulating portion 108 and the second insulating portion 110 are comprised in a single cavity within the cover 102. In another example, the cavity within the cover 102 may be split into a plurality of enclosures, for example, the cavity may comprise a first enclosure 106 and a second enclosure 104. The first enclosure 106 and second enclosure may be formed by a separating material located within the cavity.

The first enclosure 106 and second enclosure 104 may form sealed enclosures or put another way the first enclosure 106 and the second enclosure 104 may be sealed from each other. In one example, the first insulating portion 108 is located in the first enclosure 106 and the second insulating portion 110 is located within the second enclosure.

In one example, the first insulating portion 108 and the second insulation portion 110 are formed of a lightweight synthetic insulation fibres, such as Climashield®. In one example, the the second insulating portion 110 has a greater through-thickness compared with the first insulating portion 108. For example, the second insulating portion 110 may have a through thickness of between approximately 30 to 45 mm, more preferably 35 to 40 mm and the first insulating portion 108 may have a through thickness of between approximately 12 to 18 mm, more preferably 14 mm to 16 mm. In one example, the first insulating portion 108 and the second insulating portion 110 are comprised of different insulation materials and have different thicknesses. By through-thickness, it is meant the dimension that extends into the page in FIG. 1, i.e. the smallest dimension of the insulation.

In one example, the first insulating portion 108 and the second insulating portion 110 comprise a different number of layers of insulation. For example, the first insulation portion 108 may comprise between one to three layers of insulation and the second insulation portion 110 comprises between four to six layers of insulation. In one example, the first insulation portion 108 comprises two layers of insulation and the second insulation portion 110 comprises five layers of insulation. The layers of insulation may be arranged in a stacked arrangement.

In one arrangement, the first insulation portion 108 comprises a first cell of folded insulation and the second insulation portion 110 comprises a second cell of folded insulation. The cells maximise the insulating value as opposed to sewing it into smaller cells, which is often seen in commercial quilts.

In one example, the insulating material of the first insulating portion 108 and/or second insulating portion 110 may be in a substantially uncompressed state when not in use.

In one example, the blanket 100 has a substantially rectangular shape when laid out flat and viewed on plan. The blanket 100 has a longer length compared with width. In an arrangement, the first insulation portion 108 and the second insulating portion 110 are arranged sequentially lengthways and abut each other towards a central axis of the blanket 100. In other words, the first insulation portion 108 and the second insulation portion 110 both extend across substantially the full width of the blanket 100, but each extend approximately half-way across the length of the blanket 100.

In one example, the first insulating portion 108 is substantially rectangular shaped when view on plan and the second insulating portion 110 is substantially rectangular shaped when viewed on plan.

The first insulating portion 108 may have a length of between 500 mm to 1100 mm, preferably 800 mm. Further, the first insulating portion 108 may have an average width of between 200 mm and 500 mm, preferably 350 mm. The width of the first insulating portion 108 may vary across its length, for example, the width may be approximately 400 mm where it abuts the second insulating portion 110 and taper to a width of approximately 250 mm. In one example, the blanket includes a foot flap configured to be joined to the first insulating portion 108 which is configured to be folded under a patient's feet in use. The foot flap (not shown) may have a length of approximately 400 mm.

The second insulating portion 110 may have a length of between approximately 500 mm to 1100 mm, more preferably 800 mm. In one example, the second insulating portion 110 has an average width of between approximately 300 mm and 600 mm, preferably 450 mm. In one example, the width of the second insulating portion includes a tapered region and tapers towards the first insulating portion 108. For example, the second insulting portion 110 may taper from a width of approximately 490 mm to 400 mm.

In one example, the first insulating portion 108 is tapered in a direction away from the second insulating portion 110. The first insulating portion 108 may have a smaller cross-section than the second insulating portion 110 in a direction perpendicular to a longitudinal axis 112 of the blanket 100.

The cover 102 may be generally complimentary in shape, or contain areas that are complimentary in shape, with the shape of the first 108 and/or second 110 insulating portions. This reduces the amount of cover 102, or cover material, required to house the first 108 and second insulating portions, and thereby facilitates in the production of a small and lightweight blanket 100. This may also allow for a snug/tight fit between the cover 102 and the first insulating portion 108 and the second insulating portion 110 and thereby prevent displacement of the insulating portions 108/110 within the cover 102. Furthermore, this arrangement helps to reduce the amount of empty cavity space within the cover 102 and thereby mitigate heat loss from the cover 102.

The blanket 100 may further comprise two flaps 114 on opposing sides of the blanket 100. The flaps 114 extend outwardly away from a periphery of the second insulating portion 110. The flaps 114 may be substantially rectangular in shape, although other shapes are envisaged such as square, semi-circular, or polygonal. Each of the flaps 114 comprise a third insulating portion 116. In use, the flaps 114 are arranged around the sides and underneath the patient to provide insulation to a patient's abdomen, specifically a patient's kidneys.

The third insulating portion 116 may be configured to provide greater insulation than the first insulating portion 108. That is to say that the blanket 100 works to provide more insulation to the patient's torso compared with the patient's legs.

In one example, each of the flaps 114 form sealed compartments.

In one example the third insulating portion 116 has a greater through-thickness compared with the first insulating portion 108. For example, the third insulating portion 116 may have a through thickness of between approximately 30 to 45 mm, more preferably 35 mm to 40 mm and the first insulating portion 108 may have a through thickness of between approximately 12 mm to 18 mm, more preferably 14 mm to 16 mm.

In one example, the first insulating portion 108 and the third insulating portion 116 comprise a different number of layers of insulation. For example, the first insulation portion 108 comprises between one to three layers of insulation and the third insulation portion 116 comprises between four to six layers of insulation. In one example, the first insulation portion 108 comprises two layers of insulation and the third insulation portion 116 comprises five layers of insulation. The layers of insulation may be arranged in a stacked arrangement.

The cover 102 may be generally complimentary in shape, or put another way contain areas that are complimentary in shape, with the shape of third insulating portion 116 as discussed previously. This reduces the amount of cover 102, or cover material, required to house the third insulating portion 116, and thereby facilitates in the production of a small and lightweight blanket 100. This may also allow for a snug/tight fit between the cover 102 and the third insulating portion 116 and thereby prevent displacement of the third insulating portion 116 within the cover 102. Furthermore, this arrangement helps to reduce the amount of empty cavity space within the cover 102 and thereby mitigate heat loss from the cover 102.

In one example, the insulating material of the third insulating portion 116 may be in a substantially uncompressed state when not in use.

The blanket 100 may further comprise a neck and/or head flap 118. The neck and/or head flap 118 may extends outwardly from a periphery of the second insulating portion 110. The neck and/or head flap 118 may be substantially rectangular in shape and is suitably dimensioned to be wrapped about a patient's neck and/or a patient's head, although other shapes are envisaged such as cylindrical. The neck and/or head flap 118 comprises a fourth insulating portion 120. The neck and/or head flap 118 may form a sealed compartment. The neck and/or head flap 118 may further comprise a pair of drawstrings 121 to allow a user to tighten the neck and/or head flap 118 around the patient's neck and/or head.

In another example, the first insulating portion 108 and the fourth insulating portion 120 are comprised of the same type of insulating material, but the fourth insulating portion 120 has a greater through-thickness compared with the first insulating portion 108. For example, the fourth insulating portion 120 may have a through thickness of between approximately 30 mm to 45 mm, more preferably 35 mm to 40 mm and the first insulating portion 108 may have a through thickness of between approximately 12 mm to 18 mm, more preferably 14 mm to 16 mm.

In one example, the first insulating portion 108 and the fourth insulating portion 120 are comprised of different insulation materials and have different thicknesses.

In one example, the first insulating portion 108 and the fourth insulating portion 120 comprise a different number of layers of insulation. For example, the first insulation portion 108 comprises between one to three layers of insulation and the fourth insulation portion 120 comprises between four to six layers of insulation. In one example, the first insulation portion 108 comprises two layers of insulation and the fourth insulation portion 120 comprises five layers of insulation. The layers of insulation may be arranged in a stacked arrangement.

The cover 102 may be generally complimentary in shape, or put another way contain areas that are complimentary in shape, with the shape of fourth insulating portion 116 as discussed previously. This reduces the amount of cover 102, or cover material, required to house the fourth insulating portion 116, and thereby facilitates in the production of a small and lightweight blanket 100. This may also allow for a snug/tight fit between the cover 102 and the fourth insulating portion 116 and thereby prevent displacement of the fourth insulating portion 116 within the cover 102. Furthermore, this arrangement helps to reduce the amount of empty cavity space within the cover 102 and thereby mitigate heat loss from the cover 102.

In one example, the insulating material of the fourth insulating portion 120 may be in a substantially uncompressed state when not in use.

In one example, the blanket 100 may further comprise at least one thermal pad located within the cover 102, the at least one thermal pad being operable to provide warmth to the patient's body. Suitably, the thermal pad is a thermal gel pad. The thermal pads may be located adjacent the first 108 and/or second 110 insulating portions. Alternatively, or in addition to, the thermal pads may be located adjacent the third 116 and/or fourth 120 insulating portions.

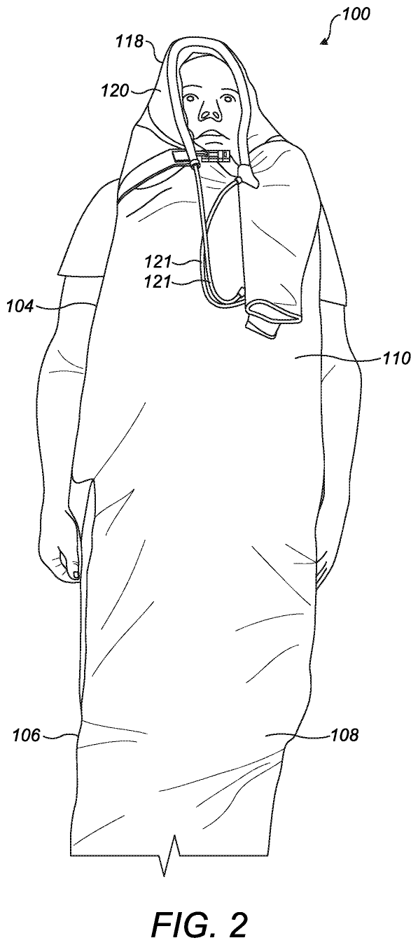

FIG. 2 shows the portable emergency blanket 100 in use when placed over the patient. The second insulating portion 110 is arranged to cover, and thereby insulate, the patient's torso, i.e. their shoulders to the groin area. The first insulating portion 108 is arranged to cover, and thereby insulate, the patient's legs and feet. In one example, flaps 114 are arranged around the sides of the patient to provide insulation to a patient's abdomen, specifically a patient's kidneys. The neck and/or head flap 118 is arranged to cover, and thereby provide insulation to, the neck and top of the patient's head. The neck and/or head flap 118 may however be used in other arrangements, for example wrapped around the patient's neck whilst leaving the head exposed.

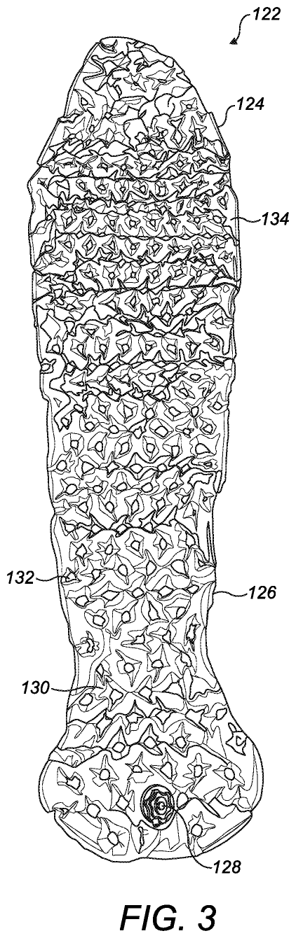

FIG. 3 shows a portable emergency inflatable mattress 122 for providing thermal insulation to a patient, the inflatable mattress 122 comprises a first region 124 to support the patient's head in use, a second region 126 to support the patient's torso and legs in use, and a one-way air valve 128, although other types of air valves are envisaged. The first region 124 and second region 126 may each comprise a plurality of cells 130 for receipt of air. The cells 130 are configured to inflate upon the receipt of air.

In one example, the first region 124 and the second region 126 are arranged directly adjacent to each other. The cells 130 in the first region 124 are typically larger than the cells in the second region 126. Suitably, the average cross-sectional dimension of the cells 130 in the first region 124 when fully inflated is between 10 cm to 16 cm, even more suitably between 12 cm to 13 cm. Suitably, the average cross-sectional dimension of the cells 128 in the second region 126 when fully inflated is between 5 cm to 9 cm, even more suitably between 7 cm to 8 cm.

The cells are partially defined by a plurality of welding points 132 between a front 134 and rear surface (not shown) of the inflatable mattress 122, i.e. areas of the front 134 and rear surfaces that have been welded to one and other. Suitably, the average distance between adjacent welding points 132 in the first region 124 is between 5 cm to 9 cm. Suitably, the average distance between adjacent welding points 132 in the second region is between 2 cm to 4 cm. The welding points 132 of the first 124 and/or second 126 regions form a repeating hexagonal pattern, however other types of repeating patterns may be envisaged such as square, rectangular, or other types of polygons. The second region 126 of the inflatable mattress 122 is tapered in a direction away from the first region 124. Generally, the inflatable mattress 122 is substantially torpedo shaped, although other types of shapes (complimentary to the shape of a human body) are envisaged. The air valve 128 may be located in the first region 124.

In use, air is provided through the one-way valve 128 and into the cells 130 located in the first 124 and second 126 regions. When the inflatable mattress 122 is inflated, suitably fully inflated, the patient is placed on the inflatable mattress 122 so that the patient's head is supported by the first region 124, and the patient's torso and legs are supported by the second region 126. The portable emergency blanket 100 may then be placed over the patient as described previously.

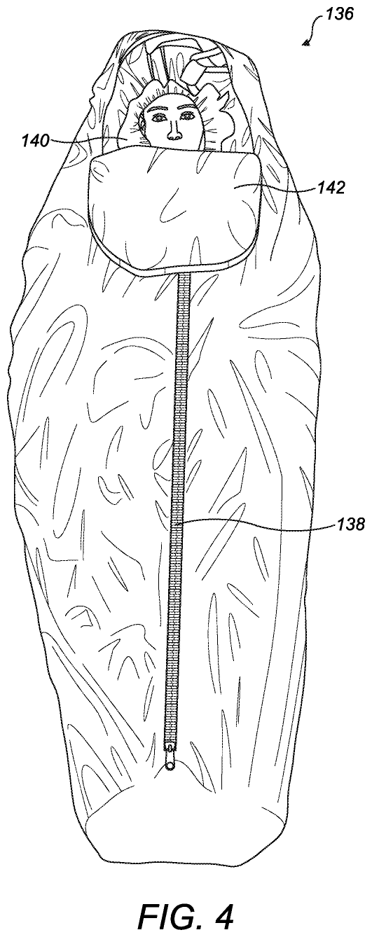

FIG. 4 shows an example of a protection sheet 136. In this example, the protection sheet 136 provides a water resilient enclosure around the patient, inflatable mattress 122 and blanket 100. The protection sheet 136 may comprise a zip fastener 138 (although over fastening means are envisaged) substantially across the length of the protection sheet 136.

The protection sheet 136 further comprises an opening 140 and a head flap 142 located above the zip fastener 138. The head flap 142 is arranged to cover the top of the patient's chest from the shoulders to nipple line.

The protection sheet 136 is configured for use with a patient who is lying on the inflatable mattress 122 and covered by the blanket 100 as described above. The zip fastener 138 is unzipped to allow access to an interior of the protection sheet 136. The patient, inflatable mattress 122 and blanket 100 are then turned on their sides to enable the protection sheet 136 to be placed over the patient. The patient is then repositioned to a lying state whilst the protection sheet 136 is positioned around the patient so that the patient's face is located adjacent the opening 140. The zip fastener 138 is then fastened.

The protection sheet 136 may be sized to substantially engulf the user, the inflatable mattress 122 and the blanket 100, in use. In one example, the protection sheet has a length of between approximately 1440 mm to 2000 mm, more preferably 1725 mm and width of approximately 800 mm to 1200 mm, more preferably 1000 mm.

In one example, the protection sheet 136 is substantially waterproof, that is to say that it provides a barrier to water. The protection sheet 136 therefore prevents the ingress of water into the enclosure around the patient.

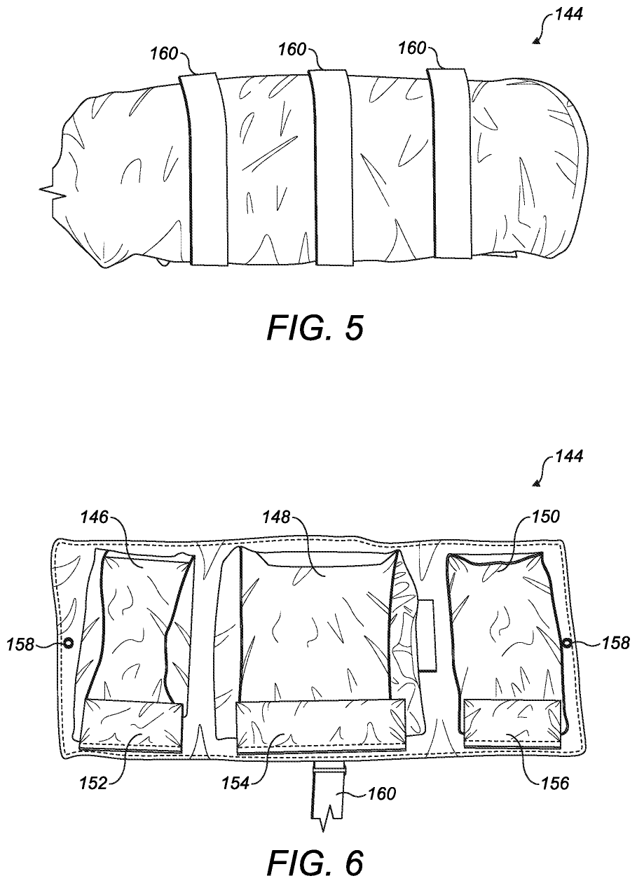

FIGS. 5 and 6 show a survival kit comprising a holder 144 comprising a first compartment 146, a second compartment 148 and optionally a third compartment 150. The first compartment 146 is dimensioned to receive and retain the inflatable mattress 122 as described previously. For example, the first compartment 146 is 340 mm by 160 mm. The second compartment 148 is dimensioned to receive and retain the blanket 100 as described previously. The third compartment 150 is dimensioned to receive and retain the protection sheet 136 as described previously. Each of the compartments 146/148/150 may comprise a cover flap 152/154/156, respectively, to cover an opening to each of the compartments 146/148/150. The second compartment 148 may have a larger dimension that the first 146 and third 150 compartments.

The holder 144 may comprise one or more securing means, the securing means being operable to receive fixtures to secure the holder 144 to the ground when the holder 144 is in the deployed configuration. The securing means may comprise a hole or loop 158 at either end of the holder 144. The holes or loops 158 are operable to receive fixtures to secure the holder 144 to the ground when the holder 144 is in a substantially flat deployed configuration (see FIG. 6). The holder may further comprise three straps 160, suitably compression straps, although one or two straps may be used. The straps 160 are of sufficient length to be wrapped around the holder 144 when the holder 144 is in a carrying configuration (see FIG. 5) to retain the holder 144 in the carrying configuration.

In use, the straps 160 are undone and the holder 144 subsequently rolled from the carrying configuration to the deployed configuration. Fixtures are then optionally placed through the holes 158 to secure the holder 144 to the ground in a substantially flat configuration. The inflatable mattress 122, blanket 100, and protection sheet 136 are then removed from each of the compartments 146/148/150 where required.

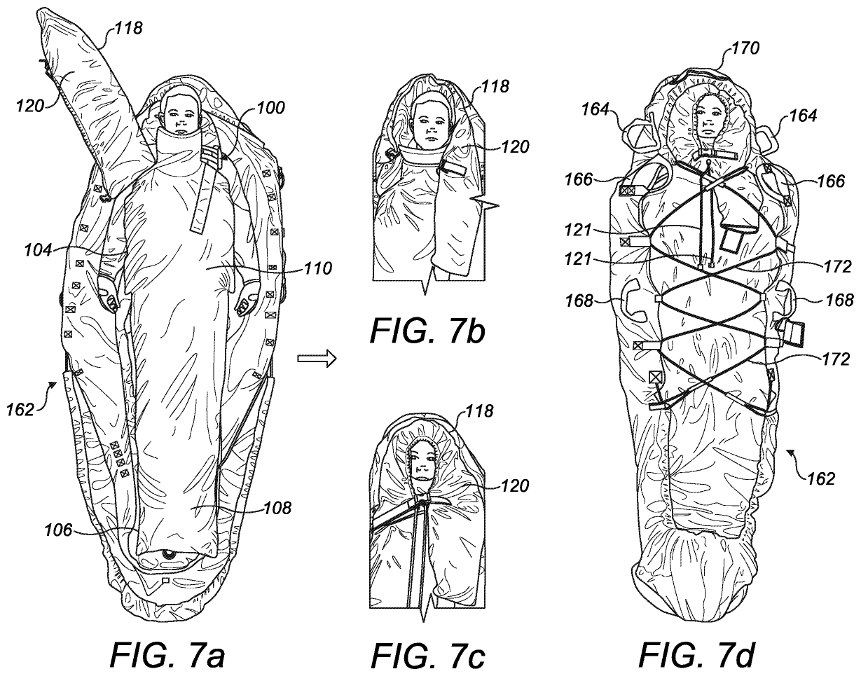

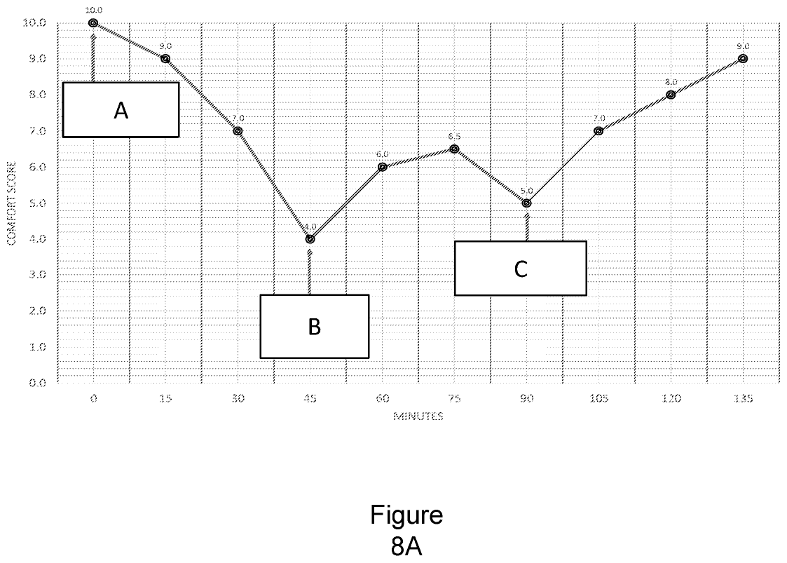

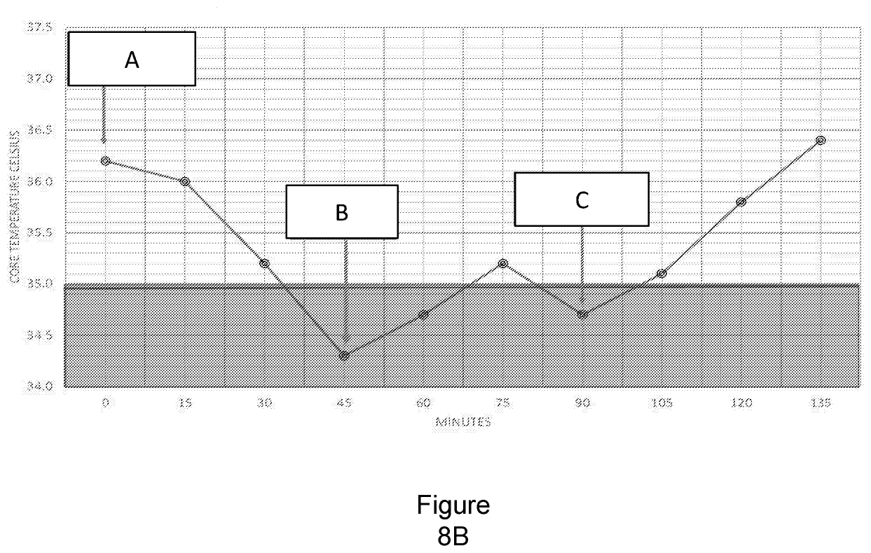

FIGS. 7a, 7b, 7c and 7d show the blanket 100 and inflatable mattress 122 in use with patient on a stretcher 162. FIG. 7a shows the stretcher 162 in an open configuration and FIGS. 7c and 7d show the stretcher 162 in a closed configuration. FIG. 7b shows the neck and/or head flap 118 wrapped around the patient's head and neck when the stretcher 162 is in the open configuration.

The stretcher 162 comprises a plurality of handles 164/166/168/170 which can be used to carry or drag the stretcher 158. The stretcher 158 further comprises a plurality of restraining straps 172 which are used to restrict movement of the patient within the stretcher 158.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

### EXAMPLES

In the following confidential study, volunteers were cooled and then rewarmed. The aim of the study was to demonstrate both the numerical decrease in the volunteers' core temperature, and objective qualitative findings from observation, as well as the volunteers' experience both in the cooling and rewarming stages.

Materials and Methods

Volunteers

| Volunteer | Sex | Age | Weight | | Height |
|---|---|---|---|---|---|
| 1 | Male | 54 | 82 | k | 1.82 m |
| 2 | Male | 53 | 71 | kg | 1.76 |

Location

In order to imitate an artic environment, the temperature of the room was maintained at 0° C. and plastic and sediment were used in the flooring. Minimal change in temperature occurred when researchers entered and exited the room.

Clothing

Volunteers wore lightweight sports shorts, t-shirts and shoes, and were soaked in water prior to entering the room.

| Examples | Equipment |
|---|---|
| 1 (comparative) | (i) External cover - uses reflective |
| | material to provide insulation |
| | (ii) Heat Cell Blanket |
| 2 (Xtract ™SR Heataver - | (i) Stretcher |
| including the blanket | (ii) Portable emergency inflatable mattress |
| of claim 1 of the | (iii) Portable emergency blanket (as |
| present inventive) | described previously) |
| | (iv) Protection sheet |
| | (v) Heat Cells |

Measurements

Tympanic temperatures taken by researchers every 15-25 minutes. Volunteers were asked for their perceived comfort on a scale of 1-10 at same time as temperature taken.

Recording Materials

Thermometer for tympanic temperature measurements. Thermal camera showing heat loss.

Results

Areas Recorded
1. Aural temperature
2. Comfort Score
3. Thermal Camera Imaging—Flir E8

### Examples

Example 2 (XtractTMSR Heatsaver) was assessed against Example 1 (a product used widely throughout US Forces).

### (1) Equipment of Example 1 Initially Used then Volunteer Transferred to Equipment of Example 2

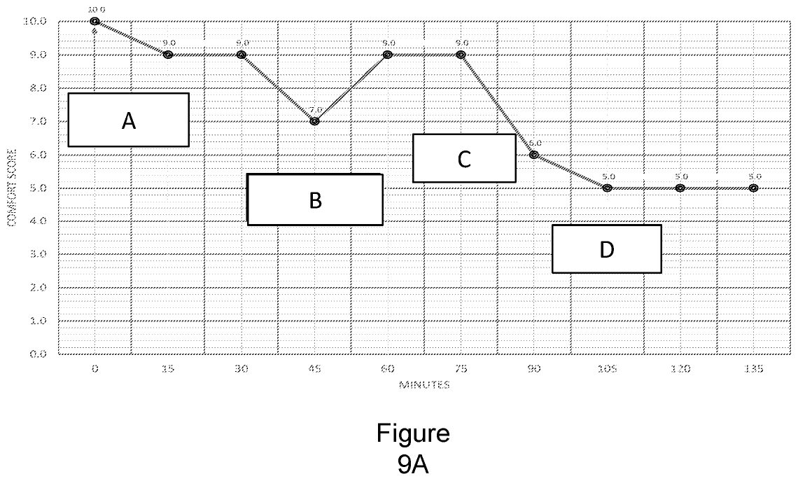

FIG. 8A shows a graph of comfort score (volunteer assessed out of 10) of a volunteer versus time (minutes) for the following experiment. FIG. 8B shows a graph of recorded core temperature (degrees Celsius) versus time (minutes).

At point A, the volunteer enters the cold room. Volunteer who struggled to recover from a hypothermic state when using equipment of Example 1. Significant shivering was noted. At point B, the volunteer uses the equipment of Example 1 (45 minutes). Volunteer then transferred to equipment of Example 2 (Xtract™SR Heatsaver) at 90 minutes at point C. Once transferred to the equipment of Example 2 volunteer's temperature and comfort scores improved.

### (2) Equipment of Example 2 Initially Used then Volunteer Transferred to Equipment of Example 1

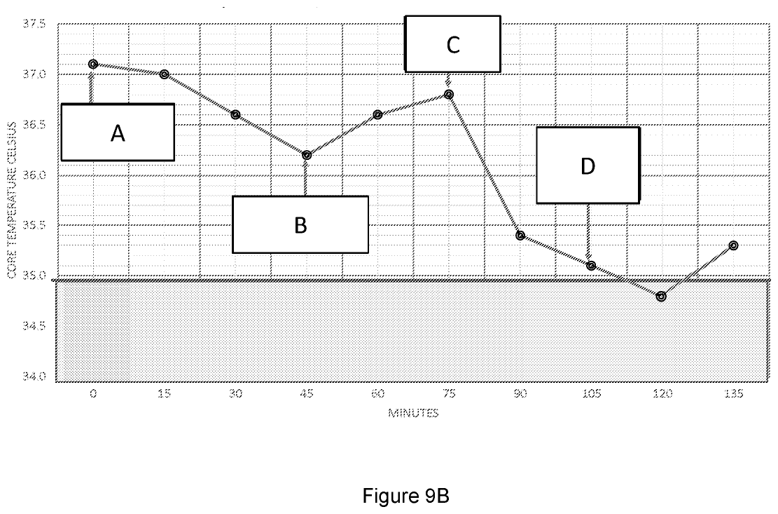

FIG. 9A shows a graph of comfort score (volunteer assessed out of 10) of a volunteer versus time (minutes) for the following experiment. FIG. 9B shows a graph of recorded core temperature (degrees Celsius) versus time (minutes).

Volunteer enters the cold room at point A (0 minutes). At point B (45 minutes in), the volunteer uses equipment of Example 2 (Xtract™SR Heatsaver). Volunteer then transferred to equipment of Example 1 at 75 minutes. At point D (105 minutes), a carry mat is inserted into the equipment of example 1. Once transferred to the equipment of Example 1 volunteer's temperature and comfort scores deteriorated.

Summary

Example 2 (Xtract™SR Heatsaver) provided a higher level of comfort and a more immediate relief. This can be shown both through the increased velocity of temperature gain and comfort level recovery.

1. A portable emergency blanket for providing thermal insulation to a patient, the blanket comprising:
a cover that houses:
a first insulating portion, the first insulating portion being configured to provide thermal insulation to a patent's legs in use; and
a second insulating portion, the second insulating portion being configured to provide thermal insulation to a patient's torso in use;
wherein the second insulating portion is configured to provide greater insulation than the first insulating portion.

2. The portable emergency blanket as claimed in claim 1, wherein the first and second insulating portions each comprise an insulating material arranged in one or more layers within the cover, and wherein the second insulating portion has a greater number of insulating material layers than the first insulating portion.

3. The portable emergency blanket according to claim 2, wherein the first insulating portion comprises two layers of insulating material and the second insulating portion comprises five layers of insulating material.

4. The portable emergency blanket as claimed in claim 2, wherein insulating material of the first insulating portion and/or the second insulating portion comprises continuous filament fibres.

5. The portable emergency blanket as claimed in claim 2, wherein the insulating material of the first insulating portion and/or second insulating portion is in a substantially uncompressed state in use.

6. The portable emergency blanket as claimed in claim 1, wherein the first insulating portion has a through thickness of between approximately 12 mm to 18 mm and the second insulating portion has a through-thickness of between approximately 30 mm to 45 mm.

7. The portable emergency blanket as claimed in claim 1, wherein the cover comprises at least two flaps on opposing sides of the blanket, each of the flaps comprising a third insulating portion and being operable to be placed around the abdomen of the patient's body.

8. The portable emergency blanket as claimed in claim 1, further comprising at least one thermal pad located within the cover, the at least one thermal pad being operable to provide warmth to the patient's body.

9. The portable emergency blanket as claimed in claim 1, wherein the cover further comprises a neck and/or head flap, the neck and/or head flap comprises a fourth insulating portion.

10. The portable emergency blanket as claimed in claim 1, wherein the first insulating portion has a smaller cross-section than the second insulating portion in a direction perpendicular to a longitudinal axis of the blanket.

11. The portable emergency blanket as claimed in claim 1, wherein the first insulating portion is tapered in a direction away from the second insulating portion.

12. A portable emergency inflatable mattress, the inflatable mattress comprising:
a first region to support the patient's head in use;
a second region to support the patient's torso and legs in use;
the first and second regions comprising a plurality of cells operable to be inflated upon receipt of air to provide a resting surface; and
an air valve;
wherein the cells in the first region are larger than the cells in the second region.

13. The portable emergency inflatable mattress as claimed in claim 12, wherein the average cross-sectional dimension of the cells in the first region when fully inflated is between 10 cm to 16 cm.

14. The portable emergency inflatable mattress as claimed in claim 12, wherein the average cross-sectional dimension of the cells in the second region when fully inflated is between 5 cm to 9 cm.

15. A survival kit comprising:
a holder including a first compartment and a second compartment;
the portable emergency inflatable mattress as claimed in claim 12, the mattress being locatable within the first compartment of the holder; and
a portable emergency blanket comprising a cover that houses a first insulating portion, the first insulating portion being configured to provide thermal insulation to a patent's legs in use; and a second insulating portion, the second insulating portion being configured to provide thermal insulation to a patient's torso in use; wherein the second insulating portion is configured to provide greater insulation than the first insulating portion, the blanket being locatable within the second compartment of the holder; and
wherein the holder is operable to be deployed from a carrying configuration to a substantially flat deployed configuration.

16. The survival kit as claimed in claim 15, further comprising a protection sheet locatable within a third compartment of the holder, the protection sheet being dimensioned to provide an enclosure around the inflatable mattress and blanket when in use by the patient.

17. The survival kit as claimed in claim 16, wherein the protection sheet comprises a water resilient material.

18. The survival kit as claimed in claim 15, wherein the holder comprises one or more securing means, the securing means being operable to receive fixtures to secure the holder to the ground when the holder is in the deployed configuration.

19. The survival kit as claimed in claim 18, wherein the securing means comprises a hole or loop.
